(19) United States
(12) Patent Application Publication
(10) Pub. No.: US 2008/0300420 A1
(43) Pub. Date: Dec. 4, 2008

(54) Process for the Preparation of Entacapone Form-A

(75) Inventors: Ghatambu Sethuram Chittoor, Chennai (IN); Rajaram Bapat Uday, Chennai (IN); Jayamani Munuamy, Chennai (IN); Naga Venkata Satya Prasanna Anjaneya Radha Krishna Murthy Muppidi, Chennai (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: SANMAR SPECIALITY CHEMICALS LTD., Chennai (IN)

(21) Appl. No.: 12/159,667

(22) PCT Filed: May 17, 2006
(86) PCT No.: PCT/IN2006/000165

§ 371 Date: Jun. 30, 2008

(30) Foreign Application Priority Data
Jan. 2, 2006 (IN) 05/CHE/2006

(51) Int. Cl. C07C 253/30

(52) U.S. Cl. 558/401

(57) ABSTRACT

A process to prepare (2E)-2-cyano-3-(3, 4-dihydroxy-5-nitrophenyl)-N,N-diethyl-2-propenamide (Entacapone) eliminating corrosive acids in the purification, with more than 99.5% purity with a Z-isomer content of less than 0.1% comprising condensation of e,4-Dihydroxy{5-nitrobensaldehyde with N,N-diethyl cyanoacetamide in the presence of a base selected from cyclic and acyclic secondary amines and a mixture of solvents, to obtain a crude product, stirring the crude product in a halogenated solvent, filtering and finally crystallization of polymorph A of Entacapone in a solvent.

### FIELD OF INVENTION

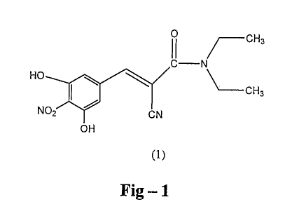

The present invention relates to a novel process for the preparation of stable crystalline polymorphic form A of (2E)-2-Cyano-3-(3,4-dihydroxy-5-nitrophenyl)-N,N-diethyl-2-propenamide (INN name Entacapone) having Formula-1 as illustrated in FIG. 1. The process is simple, efficient and amenable to large-scale manufacture. Entacapone is an inhibitor of catechol-O-methyl transferase enzyme.

### BACKGROUND AND PRIOR ART

Entacapone (formula 1) and its use in inhibiting catechol-O-methyl transferase enzyme was first disclosed in U.S. Pat. No. 4,963,590 (British Patent No. 8727854). The chemical name of Entacapone is (2E)-2-Cyano-3-(3,4-dihydroxy-5-nitrophenyl)-N,N-diethyl-2-propenamide. It is used in the treatment of Parkinson's disease as an adjunct to Levodopa/Carbidopa therapy. Parkinson's disease belongs to a group of conditions called motor system disorders, which are the result of the loss of dopamine producing brain cells. Catechol-O-methyl transferase (COMT) inhibitors allow large amount of levodopa to reach the brain, which raises dopamine levels there. Thus, they provide a more stable and constant supply of levodopa, which makes its beneficial effects last longer.

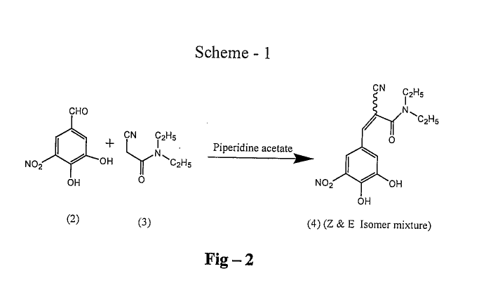

British Patent No. 8727854 describe process for the preparation of Entacapone by condensation of 3,4-dihydroxy 5-nitrobenzaldehyde with N,N-diethyl cyanoacetamide using piperidine acetate as catalyst in anhydrous ethanol as depicted in Scheme-1 shown in FIG. 2

The yield of Entacapone reported is 73%. No attempt has been made to separate the geometric isomers, which are possible for compounds like Entacapone containing carbon-carbon double bond in the structure.

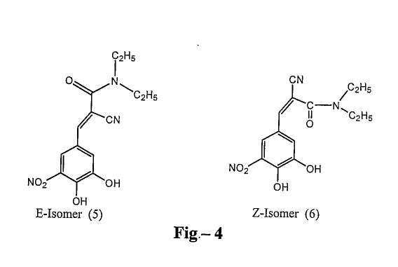

U.S. Pat. No. 5,135,950 (EP Patent No. 0426468) describes two geometric isomers E (Formula-5) and Z (Formula-6) of Entacapone and polymorphic Forms A and B of E-isomer which is shown in FIG. 4.

As per this patent, the Entacapone obtained from conventional solvent such as hydrocarbons, alcohols or esters, eg: benzene, toluene, methanol, ethanol, ethylacetate, isopropyl acetate, etc. is a very complicated mixture of different geometric isomers and/or polymorphic forms that interfere in the characterisation and standardisation of the drug substance. The bio-availability of the drug may also be influenced by polymorphism and geometrical isomerism. Further, it describes that the crystallographically pure and stable polymorphic form A of (2E)-2-cyano-3-(3,4-dihydroxy-5-nitrophenyl)-N,N-diethyl-2-propenamide (formula 5) can be obtained in good yield, when the crude product of synthesis (i.e. mixture of E and Z-Isomers) is recrystallised from lower aliphatic carboxylic acids such as formic acid or acetic acid with a catalytic amount of hydrochloric or hydrobromic acid. The said method allows large-scale production of homogenous and crystallographically pure form A of (2E)-2-cyano-3-(3,4-dihydroxy-5-nitrophenyl)-N,N-diethyl-2-propenamide (formula 5) containing a maximum of 3% and preferably a maximum of 2% of other polymorphic forms or the Z-Isomer. The main limitations of this process are—
a) Use of hazardous and corrosive acids as a solvent for recrystallisation.
b) The contamination of E-Isomer by unwanted Z-Isomer at least up to 2%.

A published patent application number WO 2005/070881 A1 describes an improved process for the manufacture of (2E)-2-Cyano-3-(3,4-dihydroxy-5-nitrophenyl)-N,N-diethyl-2-propenamide (Entacapone) polymorphic Form A containing less than 0.1% of (2Z)-2-cyano-3-(3,4-dihydroxy-5-nitrophenyl)-N,N-diethyl-2-propenamide. However, the application does not disclose the yield of (2E)-2-cyano-3-(3,4-dihydroxy-5-nitrophenyl)-N,N-diethyl-2-propenamide (Entacapone) obtained. Further corrosive acid like acetic acid is used in the final stage of the process.

Published application number WO 2005/063693 A1 describes improved process for the preparation of Entacapone, but no polymorphic form has been disclosed. Thus, a need is felt to provide for an improved process to manufacture (2E)-2-Cyano-3-(3,4-dihydroxy-5-nitrophenyl)-N,N-diethyl-2-propenamide (Entacapone) which does not make use of corrosive acid in the purification stage is efficient and is amenable to large-scale manufacture.

### OBJECT OF THE INVENTION

The objective of the present invention is to provide a simple process to manufacture entacapone 1 chemically known as (2E)-2-Cyano-3-(3,4-dihydroxy-5-nitrophenyl)-N,N-diethyl-2-propenamide an anti-catechol-O-methyl-transferase compound in crystallographically pure polymorphic form-A with Z isomer content of <0.3%.

### SUMMARY OF THE INVENTION

The present invention is directed to an improved process to prepare (2E)-2-cyano-3-(3,4-dihydroxy-5-nitrophenyl)-N,N-diethyl-2-propenamide (Entacapone, Formula-1 shown in FIG. 1 The process is efficient, does not make use of corrosive acid in the purification and is amenable to large-scale manufacture.

In one of the embodiments, the invention provides for process to manufacture crystalline polymorph A of (2E)-2-Cyano-3-(3,4-dihydroxy-5-nitrophenyl)-N,N-diethyl-2-propenamide (Entacapone) in more than 99.5% purity with Z-Isomer content of less than 0.1% comprising of—
A) Condensation of 3,4-dihydroxy-5-nitrobenzaldehyde with N,N-diethyl cyanoacetamide in presence of a base selected from cyclic and acyclic secondary amines in a mixture of ether solvent having general structure R—OR′ and a hydrocarbon solvent selected form a group consisting of heptane, hexane, petroleum ether, toluene, xylene, etc., at room temperature to 120° C.
B) Stirring of crude product obtained in Stage-A in halogenated solvent selected from methylene dichloride, ethylene dichloride, chlorobenzene, bromobenzene and chloroform, filtering the product followed by crystallisation from solvent selected from methyl alcohol, ethyl alcohol, isopropyl alcohol and acetonitrile or mixtures thereof.

### DETAILED DESCRIPTION OF THE INVENTION

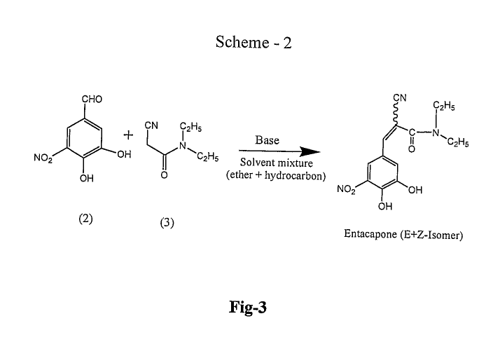

The present invention provides for a simple method for production of (2E)-2-cyano-3-(3,4-dihydroxy-5-nitrophenyl)-N,N-diethyl-2-propenamide (Entacapone) by condensing 3,4-dihydroxy-5-nitrobenzaldehyde 2 with N,N-diethyl cyanoacetamide 3 in the presence of base and a solvent mixture as shown in the Scheme-2. Shown in FIG. 3.

The process is described in more detail below:—

3,4-dihydroxy benzaldehyde (2) is condensed with N,N-diethyl cyanoacetamide (3) in presence of a base selected from a group of cyclic and acyclic secondary amines in mixture of solvents selected from a group consisting of ether solvents represented by general structure R—OR′ and a hydrocarbon solvent selected from a group consisting of heptane, hexane, petroleum ether, toluene, xylene, etc., at room temperature to 120° C.

Some of the non-limiting examples of cyclic and acyclic amines that can be used as base in the reaction are piperidine, pyrrolidine, diisopropyl amine, 4-(dimethylamino)pyridine, di-n-butyl amine, di-t-butyl amine, diisobutyl amine, etc. The most preferred amines being piperidine and di-n-butyl amine. The instant process of this invention can be best carried out by using a mixture of solvents. One of the solvents in the mixture is ether solvent having general structure R—OR′ where R&R′ are similar or dissimilar groups or together form a ring having up to 5-carbon atoms. They represent alkyl, aryl or alkoxy alkyl radical. As used in this specification the term alkyl refers to straight or branched chain having 1 to 4 carbon atoms selected from a group consisting of methyl, ethyl, n-propyl, isopropyl, butyl, isobutyl, sec butyl, t-butyl.

The term alkoxy alkyl refers to member selected from the group consisting of substituted or unsubstituted methoxymethyl, methoxyethyl, methoxypropyl.

The term aryl means an unsubstituted or substituted aryl radical such as phenyl or tolyl.

Some of the non-limiting examples of ether solvents that can be used in the process of this instant invention are diisopropyl ether, diglyme, dimethoxy ethane, di-isobutyl ether, methyl-t-butyl ether, tetrahydrofuran, methyl tetrahydrofuran.

The second component of the solvent mixture is a hydrocarbon solvent and is selected from a group consisting of heptane, petroleum ether, hexane, toluene, xylene.

The temperature of the reaction of scheme-1 can vary between room temperature to 120° C. depending upon the choice of ether solvent and hydrocarbon solvent. When solvent mixture of dimethoxy ethane and heptane is used as a solvent for reaction, the reaction is carried out at 85-95° C. temperature. The completion of reaction can be monitored by checking absence of one of the starting materials using standard techniques like in-process monitoring by HPLC. The crude product obtained after stripping of the solvent from reaction mixture is stirred with halogenated solvent selected from methylene chloride, ethylene chloride, chloroform and chlorobenzene and bromobenzene at a temperature ranging from room temperature to boiling point temperature of the solvent used in the process. For eg., if Methylene dichloride is used as a solvent, stirring is carried out at RT to 40° C. The solid obtained after filtration and drying is polymorphic form A of (2E)-2-Cyano-3-(3,4-dihydroxy-5-nitrophenyl)-N,N-diethyl-2-propenamide (Entacapone) which is crystallised from a solvent selected from methyl alcohol, ethyl alcohol, isopropyl alcohol, acetonitrile and/or a mixtures thereof to give polymorphic form A of (2E)-2-Cyano-3-(3,4-dihydroxy-5-nitrophenyl)-N,N-diethyl-2-propenamide (Entacapone) with Z-isomer content of less than 0.1% (by HPLC). The polymorphic form is characterised by IR. The IR data is given below:

Infra Red Spectrum of polymorphic form A of N,N-diethyl-2-cyano-3-(3,4-dihydroxy-5-nitro phenyl)propamide is characterised by the following peaks at about 3339, 3092, 3066, 3038, 2981, 2937, 1628, 1606, 1544, 1512, 1441, 1377, 1298, 1280, 1209, 1164, 1149, 800, 778 and 743 cm⁻¹.

The invention is illustrated further by following non-limiting examples:

### EXAMPLE-1

### Preparation of (2E)-2-Cyano-3-(3,4-dihydroxy-5-nitrophenyl)-N,N-diethyl-2-propenamide (Entacapone)

3,4-Dihydroxy-5-nitrobenzaldehyde (50 g), N,N-diethyl cyanoacetamide (76.5 g), piperidine (19.2 g), are charged to a reaction vessel containing a mixture of dimethoxy ethane (200 ml) and heptane (200 ml). The reaction mixture is refluxed for 15-25 hours till the starting material is disappeared (monitored by HPLC). The solvent was removed by distillation under high vacuum at 70-80° C. Then cooled to 25-35° C. To the crude mass in the flask was added methylene chloride (1.0 lit) at 25° C.-35° C. The mixture was stirred for 24 hours at that temperature. It is then filtered and dried and charged to reaction flask containing methanol. The mixture is charcolised, filtered and concentrated to give title compound in 75% yield (HPLC 99.78%, Z-isomer content <0.1%).

### EXAMPLE-2

3,4-Dihydroxy-5-nitrobenzaldehyde (25 g), N,N-diethyl cyanoacetamide (38.25 g), dibutylamine (10 g), are charged to in a mixture of dimethoxy ethane (100 ml) and heptane (100 ml). The reaction mixture is refluxed for 15-25 hours till the starting material is disappeared (monitored by HPLC). The solvent was removed by distillation under high vacuum at 70-80° C. Then cooled to 25-35° C. To the crude mass in the flask was added methylene chloride (500 ml) at 25° C.-35° C. The mixture was stirred for 24 hours at that temperature. It is then filtered, washed with water, dried and is charged to reaction flask containing acetonitrile. The mixture is charcolised, filtered and concentrated to give title compound (HPLC 99.7%, Z-isomer content <0.1%).

1-26. (canceled)

27. A process to prepare crystalline polymorph A (2E)-2-Cyano-3-(3,4-dihydroxy-5-nitrophenyl)-N,N-diethyl-2-propenamide (Entacapone) with at least 99.5% purity with Z-isomer content of less than 0.1% comprising the steps and sequence:—
a. Condensation of 3,4-dihydroxy-5-nitro benzaldehyde with N,N-diethyl cyano acetamide in presence of a base and a mixture of solvents to obtain a crude product,
b. Stirring of crude product obtained, in halogenated solvent and filtering to eliminate impurities and to obtain polymorph A of Entacapone
c. Crystallation of polymorph A of Entacapone in a solvent,

28. A process according to claim 27 wherein the base is selected from cyclic and acyclic secondary amines.

29. A process according to claim 27 wherein the cyclic and acyclic secondary amines are selected from a group comprising piperidine, pyrrolidine, diisopropyl amine, 4-(dimethyl amino)pyridine, di-n-butylamine, di-t-butylamine, diisobutylamine, or mixture thereof.

30. A process according to claim 27 wherein the mixture of solvents is selected from a group comprising ether solvents and from a group comprising hydrocarbon solvents.

31. A process according to claim 30 wherein the ether solvents shall have a general formula R—OR′, wherein R&R′ are similar or dissimilar alkyl or alkoxyalkyl groups of up to 4-carbon atoms or together form a ring of 5-carbon atoms.

32. A process according to claim 31 wherein the alkyl is selected from a group comprising of methyl, ethyl, n-propyl, isopropyl, butyl, isobutyl, sec butyl, t-butyl.

33. A process according to claim 31 wherein the alkoxy alkyl is selected from a group comprising of methoxy methyl, methoxy ethyl and methoxy propyl.

34. A process according to claim 27 wherein the ether solvent is selected from a group comprising, diisopropyl ether, diglyme, dimethoxy ethane, di-isopropyl ether, methyl-t-butyl ether, tetrahydrofuran and methyl tetrahydrofuran.

35. A process according to claim 27 wherein the hydrocarbon selected is selected from a group comprising of heptane, hexane, petroleum ether, toluene, xylene and mixture thereof.

36. A process according to claim 27 wherein the halogenated solvent is selected from methylene dichloride, ethylene dichloride, chlorobenzene, bromobenzene and chloroform.

37. A process according to claim 27 wherein the condensation is at room temperature to 120° C.

38. A process according to claim 27 wherein the ether solvent is dimethoxy ethane and hydrocarbon solvent is heptane.

39. A process according to claim 27 wherein the ether solvent is di-isopropyl ether and hydrocarbon solvent is heptane.

40. A process according to claim 27 wherein the ether solvent is dimethoxy ethane and hydrocarbon solvent is hexane.

41. A process according to claim 27 wherein the ether solvent is dimethoxy ethane and hydrocarbon solvent is toluene.

42. A process according to claim 27 wherein the ether solvent is dimethoxy ethane and hydrocarbon solvent is toluene.

43. A process according to claim 27 wherein the ether solvent is di-isopropyl ether and hydrocarbon solvent is toluene.

44. A process according to claim 27 wherein the solvent for crystallization is selected from a group comprising methyl alcohol, ethyl alcohol, isopropyl alcohol and acetonitrile or mixtures thereof.

45. A process according to claim 27 wherein the solvent for crystallization is methanol.

46. A process according to claim 27 wherein the solvent for crystallization is ethanol.

47. A process according to claim 27 wherein the solvent for crystallization is acetonitrile.

48. A process according to claim 27 wherein the reaction temperature is the boiling temperature of hydrocarbon solvent.
